(19) United States
(12) Patent Application Publication
(10) Pub. No.: US 2011/0117620 A1
(43) Pub. Date: May 19, 2011

(54) PROCESS FOR PRODUCING METHANE FROM PROCESS WATER AND BIOGENIC MATERIAL

(76) Inventor: Johann Rietzler, Nürnberg (DE)

(21) Appl. No.: 13/003,489

(22) PCT Filed: Jun. 30, 2009
(86) PCT No.: PCT/DE09/00932

§ 371 Date: Jan. 10, 2011

(30) Foreign Application Priority Data
Jul. 10, 2008 (DE) 10 2008 032 409.4

(51) Int. Cl. C12P 5/02; C12M 1/107; C12M 1/38

(52) U.S. Cl. 435/167; 435/290.1; 435/286.1

(57) ABSTRACT

A process for producing methane from process water and biogenic material such as that occurring in the production of sugar and ethanol, wherein at least one mixing/preliminary tank is supplied with process water and biomass, and optionally with washing and/or fresh water and/or substrate water to produce a mash, the mash is set to a suitable pH value and temperature. The mash is transferred into at least one bioreactor with anaerobic methane bacteria for biogas production, the biogas developed is extracted and the biodegraded fluid is drawn off. A biogas plant for performing this process has at least one mixing or preliminary tank for mash provided with a heat exchanger system, and at least one downstream biogas reactor having bacteria producing biogas. The biogas plant is used for producing biogas and storing waste and process water and by-products from the sugar and ethanol production operation.

### BACKGROUND OF THE INVENTION

1. Field of the Invention

The invention relates to a process for the production of methane from process water and biogenic material, in particular those, which accrues during sugar and ethanol production; a biogas facility as well as its use.

2. Description of Related Art

The fermentation of biological materials is known since long. Due to most diverse developments, single, two or multi-stage processes were developed. Besides wet fermentation developed from manure fermentation, dry fermentation is also practiced. These processes were often put to practice. German Patent Application DE 10 2004 053 615 A1 and corresponding U.S. Pat. No. 7,854,840 B2 disclose a process for the production of methane from biologically available organic ingredients of waste water. This process uses a percolator as hydrolysis stage and a bioreactor with methane bacteria for biogas generation. Once the percolator fluid is stored, it can then be transferred, if new gas is required, into the bioreactor for producing gas. This process can be improved, since it cannot make the necessary capacity available with large quantities of waste and process water.

A further problem of the hitherto known process lies in the fact that, if a dormant season exists or no process water is available, it cannot be sustained for several months because of lack of necessary storage capacity. If methane bacteria remain unattended, they die. Further, no biogas production is then possible.

### SUMMARY OF THE INVENTION

The invention is directed to solving the problem of producing methane from large quantities of waste and process water, in particular from agricultural production, like sugar and ethanol production, which in the past were distributed untreated on agricultural land. Since the waste and process water quantities are not available throughout the year, a year-long operation of biogas production must be made possible with biomass during shutdown of production of sugar and ethanol.

The problem is solved by a process with the features described herein as well as by a biogas facility with the features as described.

This process has the advantage that it is based on a simple technology for bio-degrading of material, which was hitherto distributed usually untreated on the areas under cultivation. Thus, according to the said invention, a degradation of waste and process water and by-products from the production of sugar and ethanol and biogenic material with all-season operation of the biogas reactor is possible, with which periodically accruing waste and process water is treated in large quantities of over 10,000 m3 per day and in the remaining periods by using fresh water and recycling of substrate water in combination with renewable raw materials or organic material, it is possible to exploit biogas and to use it for energy production throughout the year. Cleaned waste water is made available just like the accruing sludge for fertilization and irrigation. By use of fresh water and recycling of substrate water for compensating the delivery fluctuations and for leveling the biogas operation, a recycling of substrate water as well as re-circulating sludge in/or between the preliminary tank and the biogas reactors may be foreseen. The water accruing here can likewise be used for irrigation purpose. Thus, a demand based irrigation lasting all throughout the year as well as a continuous control of biogas accrual is made possible and the biogas requirement, for example, for the generation of electricity or heat, continuously and/or in peak and/or low load periods, is regulated accordingly. While in case of known facilities, control of biogas production does not take place or only in closer periods for adjustment according to consumption, a leveling of the biogas exploitation and production of electricity and heat can take place with the help of a decomposition process according to the invention. As biogenic material are considered substances stemming from organisms like plants, animals, single-cell organisms, viruses etc., in particular, distiller's waste and process water and further natural by-products of the production of renewable raw materials, like, for example: washing water, fusel oil and filter cakes or sugarcane from ethanol and sugar extraction, bio-waste, green waste, industrial waste, food waste, agricultural waste, renewable raw materials, alkaline fermenter fluid, waste water from starch production from potatoes, peas and beans etc. and other similar materials. Preferably, waste/process water as well as biogenic material and the above mentioned byproducts are stored in a suitably sized buffer reactor and/or preliminary tanks (e.g., 24 h buffer) for liquids in a predetermined proportion to one another, since the methane reactions subsequently accomplished by bacteria in an anaerobic biogas reactor for biogas production are in the hourly range. As methane bacteria are thermally sensitive, the maximum temperatures possible for the respective bacteria strains should not be exceeded, for example approx. 55° C., better even approx. 37° C. in the methane gas reactors.

In a preferred embodiment, the waste and process water resulting under process temperature of up to 95° C. and/or waste/process water heated to approx. 55° C. or substrate or fresh water with biomass and/or biologically degradable renewable raw materials or by-products, such as fusel oil and (sugarcane) filter cakes as well as washing water are mixed in a tank and the thus produced mash yielded after a reaction period up to 24 hr with venting of nascent carbon dioxide is distributed extensively in the base region of a lagoon facility. Thanks to the homogeneous distribution of the mash as well as a circulation of biogas within biogas reactors at constant temperature conditions of approx. 55° C.±2° C. and approx. 37° C., an optimum process of continuous biogas development under degradation of organic carbon within the said thermophilic and mesophilic ranges is reached with the most extensive degradation of biogenic material within 7 to 15 days.

With the help of the process of biogas circulation with the substrate, a reduction of carbon dioxide takes place in the biogas as in the case of mesophilic operation with relative increase of the methane gas portions and an increase in calorific value, in line with the mixing process of the substrate. The carbon dioxide content can be further reduced by the addition of milk of lime or alkaline washing water. Since the process water is not available all year round in the same quantity, the lagoon containers are operated module-like with several basins, which are interconnected with pipelines. Subsequently, process water of ethanol/sugar production, also known as Vinhaca, in accordance with the invention is described subsequently, wherein it is not limited to this embodiment under any circumstances. Accruing waste and process water, e.g., from ethanol sugar production, coming out of the distillery has a temperature of up to 95° C. While mixing with small chaffed plant raw materials in a ventilated reaction vessel, the leaf structure of the plant is effectively destroyed, so that a quick availability of biodegradable materials can take place in the anaerobic biogas reactor downstream. Thus, the degradation process of plant raw materials, which runs up to 53 days in conventional biogas reactors, can be reduced effectively. By use of a heat exchanger, a temperature of the liquid in the reaction vessel can be reduced to approx. 55-58° C. before introducing it into the anaerobic biogas reactor. At the above-said temperatures, hydrolysis and acidification take place in an accelerated manner in the reaction vessel as well as CO₂-formation, whereby the CO₂ under the aerobic conditions present in the reaction vessel can be partially expelled by air supply.

In case of a reduced offer of waste and process water, fresh or substrate water at approx. 52-57° C. is mixed and used with the chaffed plant raw materials as well as the by-products mentioned above. After a residence time of max 24 hr in the reaction vessel, the mash is fed to a biogas reactor in lagoon form, in which under anaerobic conditions the actual methanogenesis takes place by means of methane bacteria leading to the formation of methane and carbon dioxide. The methane bacteria can be immobilized on carriers or free. The lagoon containers are provided with an air roof, whereby the available free area serves as gas storage. Preferably, the biogas present in the gas storage space is partly pressed into the floor level of the lagoon container, thus a better circulation of the substrate as well as methane discharge and better biogas development are achieved by removing the inhibition of the reaction equilibrium. Further, alkali, e.g., milk of lime, can be dosed for further bonding of carbon dioxide into the lagoon containers. The lagoon facility can have at least two basins connected by pipelines, which would suffice for an inflow varying by up to over 10,000 m³ per day of accruing waste/process water. The reactor is kept continuously operating at approx. 55° C. and/or approx. 37° C. by the heat exchanger system as described.

Owing to the circulation of biogas in the lagoon containers, a settling process of sedimentation and/or floating particles takes place despite the mixing of the substrate. These are withdrawn at the floor level by means of screw pumps from the reactor and made available after the passage through filter belt presses or comparable drainage mechanisms, such as centrifuges for fertilizing and the filtration and/or substrate water are recycled between the reaction vessel and the lagoon facility. Further, the lagoon containers comprise an overflow, through which the degassed substrate water is cleared of sludge, withdrawn and made available for fertilizing purpose or recycled as substitute for waste/process water. The fermentation of biogas is preferably carried out by means of bacteria. In doing so, the fermentation is preferably carried out using a bacteria matrix of several bacteria strains. Depending upon the bacteria strain, these different materials can ferment and can also provide another ratio of methane/CO₂. Using the aforesaid heat exchanger system, the biogas reactor can be heated, in particular, externally. Thus, a constant temperature can always be maintained in the biogas reactor. This lies favorably at approx. 55° C. and approx. 37° C.

The application of the input system for bringing biodegradable materials into the biogas reactor and the recycling of substrate water has the further advantage that continuous operation of the biogas facility is possible at any time. Further, adaptation to the respective material accrual and/or the energy demand is possible. During degradation of biogenic materials, acids are formed, thus the mixing tanks as well as the biogas reactor are made preferably acid-resistant. The process can be used for other processes with the accrual of extremely acidic process water in the biogas reactor. For example, food packaging industry yields excessive waste water flow with organic load that is usually strongly acidic, and there are solid wastes, on the other.

With demand-based use of solid biogenic and plant raw materials in combination with the supply of fresh water and the recycling of substrate water, the seasonally varying accrual of acidic waste water can be balanced, so that in times of low waste water accrual still a good biogas production is available. The biogas reactor can be gas-tight and functions, preferably, according to one of the reactor principles usual in case of wastewater technology (UASB [Upflow Anaerobic Sludge bed], biogas consists of methane (CH₄) [50-85 Vol-%], carbon dioxide (CO₂) [15-50 Vol-%] as well as traces of oxygen, nitrogen and trace gases (among other things, hydrogen sulphide). With the said microbiological degradation process, biogas is generated with a high methane content of between 60 and 80 Vol-%. It can be used, inter alia, directly for heating or by means of a combined heat and power unit for co-generation of electricity and heat.

The generation of gas takes place via anaerobic fermentation of organic materials. For increasing the biogas yield, co-fermented material is frequently used (for example, renewable raw materials or waste from the foodstuffs industry). The fermented organic material can be agriculturally used afterwards as high-quality fertilizer. In a preferred embodiment of the invention, the storage buffer and/or mixing/premixing tank may be aerated. By mixing with air, biogas can easily lead to explosive mixtures; therefore the production and storage are subject to special safety regulations. Preferably, the mixing tank has a volume of approx. 50 to 100% of the daily accruing waste/process water or fresh water. It serves for mixing the supplied material flows as mentioned above (liquids with biomass and by-products from the sugarcane processing) and is provided with a heat exchanger system. Further, ventilation is provided for expelling carbon dioxide. The biomass is finely cut by a chaff cutter and supplied through a conveying system to the mixing tank. The danger of explosion can be precluded according to the invention, since biogas production takes place exclusively in the lagoon facility, which is provided with an air roof, that simultaneously serves as gas storage space and further, the operation takes place under anaerobic conditions. Moreover, storage of biogas is not necessary, since the entire biogas is transferred directly to the combined heat and power plant downstream. As a safety system, an emergency flair is installed, which comes into operation in case of failure of the CHP.

The invention is described in detail by way of example only in conjunction with the accompanying drawings.

### BRIEF DESCRIPTION OF THE DRAWINGS

FIG. 1 is a schematic diagram of a simple biogas facility according to the invention for demand-based production of biogas from sugarcane wastes; and

FIG. 2 is a schematic diagram of a further biogas facility according to the invention with several biogas reactors.

### DETAILED DESCRIPTION OF THE INVENTION

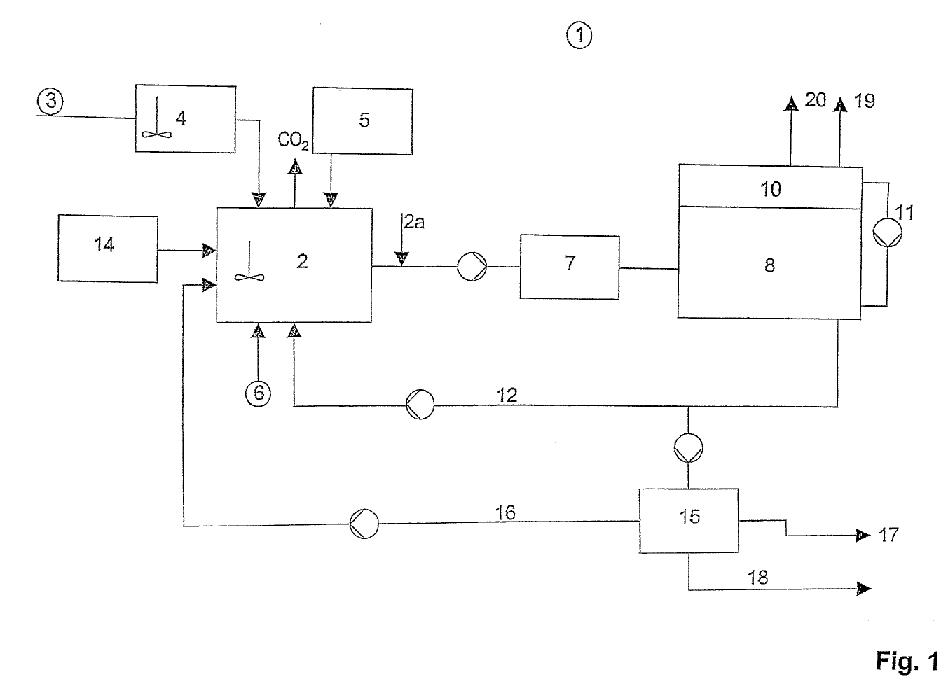

As shown in FIG. 1, biological material is transferred from a chaff cutter 3 into a conveying system 4. From the conveying system 4, the comminuted biomaterial is transferred to the reaction and mixing tank 2 according to demand. Further, fresh/wash liquor 12—depending on demand—can be supplied to the mixing tank as well as Vinhaca (liquid with organic residue from the ethanol distillation of fermented sugarcane—approx. 3-10% organic materials, and 1% mineral solids, remainder water—about 4-5 wt. % dry materials). Finally, filtration water from the biogas reactor 8 can be led via a return line of 12, 16 into the reaction and mixing tank. With fresh Vinhaca from production, a temperature of up to 95° C. as well as a pH value within the acid range (approx. 4-5.0 pH) prevails in the Vinhaca bunker 5. The wash liquor or washing water has a pH value in strongly alkaline solution, preferably around pH 10-12. In the tank 2, these material flows are mixed with mash and adjusted—preferably automatically—to a weakly acidic pH value of approx. 5. To this end, a pH value sensor (not shown) can be provided in the reactor 2. The so-called mash is brought by a heat exchanger 7 to a suitable temperature for the methane bacteria in the biogas reactor 8. If necessary, the pH value can be measured and readjusted again via line 2a.

In the biogas reactor 8, there are free or immobilized methane bacteria. They decompose the ingredients of the aqueous solution with up to approx. 12 wt % drying materials to CO₂ and methane gas. Methane is collected in the gas storage space 10 and withdrawn via line 19. For excessive methane, an emergency vent 20 is available which can lead to a buffer tank or an emergency flare. From the gas storage space, a recirculation line 11 leads to the base of the bioreactor 8 in order to encourage the reaction to methane by the recycling of gas and to expel the reaction-inhibiting CO₂. A part of the biogas reaction solution during reaction in the preliminary/mixing tank 2 can be recycled. The converted bioreactor solution can be taken out via the sludge withdrawal in filtration water 16, which can either be recycled into the mixer 2 or utilized otherwise by being withdrawn via line 18, whereby the sludge can be used further.

The fact that biological material is constantly delivered to reactor 2 by the chaff cutter 3 via the conveying system 4, it is possible to maintain methane production even during the absence of Vinhaca because of a shutdown of Vinhaca production. Thus, it is important that the methane bacteria are kept alive and a further production of biogas is possible.

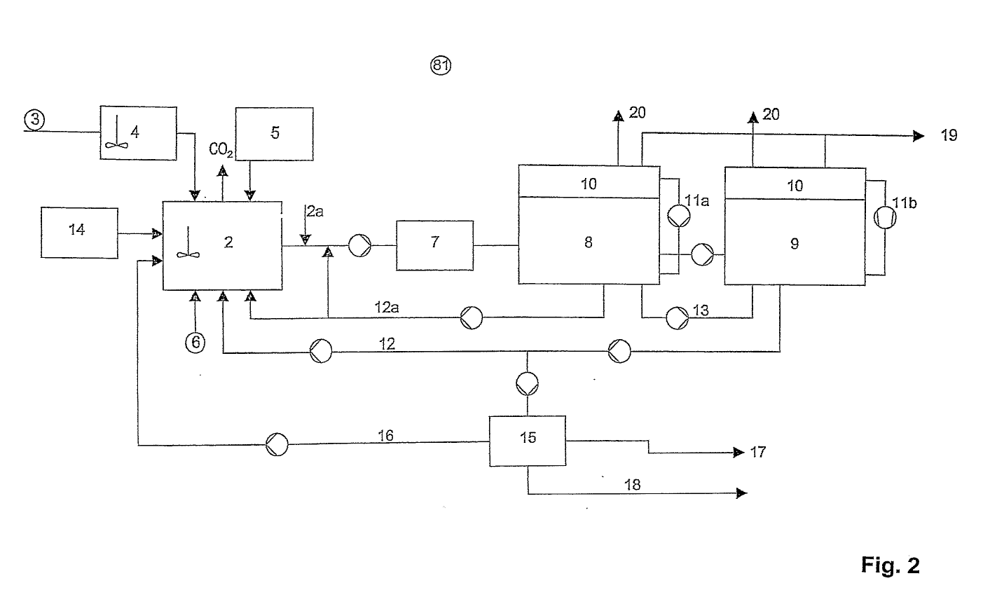

FIG. 2 shows a more complex plant 81 for biogas production from the same raw materials, as explained in FIG. 1. Therein biological material from chaff cutter 3 and a feed hopper 4, Vinhaca from sources 4, 5 and wash liquor or fresh water from supply 14 are fed in such proportions so that the dry material content of the aqueous suspension lies at about 12%. Further, air is supplied to reactor 2 from air source 6 to expel from the mash CO₂ which inhibits the methane formation reaction. Return lines 12, 12a from the biogas reactors 8, 9 are provided to the reactor 2. Using a suitable heat exchanger 7, the mash is then brought to an optimum temperature and pH value suitable for methane bacteria in biogas reactor 8 and then transferred to the biogas reactor 8. In this are present the methane bacteria, which work in the temperature range of 55° C.

The residue from the methane bioreactor 8 is transferred to a further bioreactor 9 that is connected to it in series, and in which a further methane bacteria strain is kept, which operates at a temperature of about 37° C. and has another profile for processing. The biogas facility 1 can be operated in such a manner that the aerobic mixing tank and the anaerobic biogas production cycle are strictly separated from each other. Thus, it is guaranteed that no unsafe quantity of free biogas (methane) is present. This leads to an improved operating safety of the entire plant.

While special embodiments of the invention were shown and described, various deviations and alternative embodiments are obvious to the expert in the field. Therefore, the invention is limited only by the scope of the claims.

1-18. (canceled)

19. Process for the production of methane from process water and biogenic material selected from the group consisting of sugarcane leaves, filter cakes of sugar production, fusel oil, and washing water from sugar and ethanol production, comprising the steps of:
loading at least one mixing or preliminary tank with at least process water and biomass and forming a mash therewith,
setting the mash to a pH value and temperature adapted for producing methane from the mash,
after setting the pH value and temperature of the mash, transferring the mash to at least one bioreactor having anaerobic methane bacteria and producing biogas therein,
withdrawing the biogas produced, and
taking out biologically degraded liquid from the at least one bioreactor.

20. Process according to claim 19, wherein biogas is re-circulated in the at least one bioreactor.

21. Process according to claim 19, wherein an alkali selected from the group consisting of at least one of an alkaline solution, alkaline washing water from sugar-ethanol production, milk of lime, ammonia is introduced into the at least one bioreactor for increasing of pH value by lowering CO₂-content as biogas is formed.

22. Process according to claim 19, comprising the further step of performing aerification in the mixing or preliminary tank to obtain aerobic conditions and to expel carbon dioxide.

23. Process according to claim 19, wherein the mixing or preliminary tank is operated aerobically.

24. Process according to claim 19, wherein fermentation to biogas is performed with at least one of added and immobilized bacteria.

25. Process according to claim 19, wherein said loading step further comprises the addition of at least one of washing water, fresh water, and substrate water under production of mash in aerobic conditions leading to hydrolysis in weak acids.

26. Biogas facility for the production of methane from process water and biogenic material, comprising at least one mixing or preliminary tank, a heat exchanger system connected to said at least one mixing or preliminary tank for receiving mash therefrom and at least biogas reactor with bacteria located downstream of heat exchanger system producing biogas from mash received from the heat exchanger system.

27. Biogas facility according to claim 26, wherein the at least one biogas reactor is a lagoon container with a system for distribution of supplied mash to a substrate.

28. Biogas facility according to claim 27, further comprising a system for circulation of biogas in the substrate.

29. Biogas facility according to claim 26, further comprising a system for separating sludge and fermented substrate and recycling of substrate or filtration water to the at least one biogas reactor.

30. Biogas facility according to claim 26, further comprising at least one system for dewatering of accruing sludge.

31. Biogas facility according to claim 27, wherein the lagoon container is multi-chambered with a gas storage in an air roof.

32. Biogas facility according to claim 27, wherein the lagoon container is made of an acid-resistant material.

33. Biogas facility according to claim 26, wherein the at least one biogas reactor is has a temperature controller.

34. Biogas facility according to claim 26, wherein the biogas reactor is gas-tight.

35. Biogas facility according to claim 27, wherein the mixing or preliminary tank is connected to a source of air for aeration thereof.
